(19) United States
(12) Patent Application Publication
(10) Pub. No.: US 2012/0103835 A1
(43) Pub. Date: May 3, 2012

(54) SYSTEM AND/OR METHOD OF VALIDATING METERED BLOOD GLUCOSE FOR GLUCOSE SENSOR CALIBRATION

(76) Inventors: Bradley Liang, Bloomfield Hills, MI (US); Kenneth W. Cooper, Newhall, CA (US); Raghavendhar Gautham, Los Angeles, CA (US); Rajiv Shah, Rancho Palos Verdes, CA (US)

(21) Appl. No.: 13/282,166

(22) Filed: Oct. 26, 2011

Related U.S. Application Data
(60) Provisional application No. 61/407,876, filed on Oct. 28, 2010.

(51) Int. Cl. G01N 33/66; G01N 27/327

(52) U.S. Cl. 205/782; 204/403.01

(57) ABSTRACT

The subject matter disclosed herein relates to systems, methods and/or devices for calibrating sensor data to be used in estimating a blood glucose concentration. A relationship between sensor signal values and reference readings may be used to estimate a relationship between sensor signal values and measurements of blood glucose concentration.

This application claims the benefit of priority to U.S. Provisional Patent Application No. 61/407,876, titled “System and/or Method of Validating Metered Blood Glucose for Glucose Sensor Calibration,” filed on Oct. 28, 2010, and assigned to the assignee of claimed subject matter, and incorporated herein by reference.

### BACKGROUND

1. Field

The subject matter disclosed herein relates to calibration of glucose sensors for use in glucose monitoring systems, for example.

2. Information

Over the years, body characteristics have been determined by obtaining a sample of bodily fluid. For example, diabetics often test for blood glucose levels. Traditional blood glucose determinations have utilized a painful finger prick using a lancet to withdraw a small blood sample. This results in discomfort from the lancet as it contacts nerves in the subcutaneous tissue. The pain of lancing and the cumulative discomfort from multiple needle pricks is a strong reason why patients fail to comply with a medical testing regimen used to determine a change in a body characteristic over a period of time. Although non-invasive systems have been proposed, or are in development, none to date have been commercialized that are effective and provide accurate results. In addition, all of these systems are designed to provide data at discrete points and do not provide continuous data to show the variations in the characteristic between testing times.

A variety of implantable electrochemical sensors have been developed for detecting and/or quantifying specific agents or compositions in a patient's blood. For instance, glucose sensors are being developed for use in obtaining an indication of blood glucose levels in a diabetic patient. Such readings are useful in monitoring and/or adjusting a treatment regimen which typically includes the regular administration of insulin to the patient. Thus, blood glucose readings improve medical therapies with semi-automated medication infusion pumps of the external type, as generally described in U.S. Pat. Nos. 4,562,751; 4,678,408; and 4,685,903; or automated implantable medication infusion pumps, as generally described in U.S. Pat. No. 4,573,994. Typical thin film sensors are described in commonly assigned U.S. Pat. Nos. 5,390,671; 5,391,250; 5,482,473; and 5,586,553. See also U.S. Pat. No. 5,299,571.

### BRIEF DESCRIPTION OF THE FIGURES

Non-limiting and non-exhaustive features will be described with reference to the following figures, wherein like reference numerals refer to like parts throughout the various figures.

FIG. 1(a) is a perspective view of an example glucose sensor system for use in accordance with an embodiment.

FIG. 1(b) is a side cross-sectional view of a glucose sensor system of FIG. 1(a) for an embodiment.

FIG. 1(c) is a perspective view of an example sensor set for a glucose sensor system of FIG. 1(a) for use in accordance with an embodiment.

FIG. 1(d) is a side cross-sectional view of a sensor set of FIG. 1(c) for an embodiment.

FIGS. 2a through 2c are diagrams showing a relationship between sampled values, interval values and memory storage values according to an embodiment;

FIG. 3 is a chart illustrating the pairing of a blood glucose reference reading with glucose monitor data according to an embodiment;

FIG. 4A is a flow diagram illustrating a process for incorporating metered blood glucose reference samples in calibration of a blood glucose sensor according to an embodiment;

FIG. 4B is a flow diagram of a process for determining whether to accept or reject a blood glucose reference sample for use in calibration of a blood glucose sensor according to an embodiment.

FIG. 4C shows plots of metered blood glucose reference samples including suspicious outliers.

FIG. 5 is a flow diagram of a process for determining whether to accept or reject a blood glucose reference sample for use in calibration of a blood glucose sensor according to an alternative embodiment.

### SUMMARY

Briefly, example embodiments may relate to methods, systems, apparatuses, and/or articles, for validating metered blood glucose samples. In one particular implementation, a method comprises: obtaining a blood glucose reference sample from a patient; based, at least in part on predefined criteria, selectively excluding the blood glucose reference sample for use in deriving a relationship between glucose sensor signal values and glucose sensor measurements based, at least in part, on temporal pairings of blood glucose reference samples with glucose sensor signal values. In one particular example implementation, excluding the blood glucose reference sample further comprises: determining that the blood glucose reference sample deviates from a trend. In another particular example implementation, determining that the blood glucose reference sample deviates from the trend further comprises: correlating the blood glucose reference sample with a glucose sensor measurement; and determining whether a relative difference between a value of the blood glucose reference sample and the glucose sensor measurement exceeds a threshold. In yet another implementation, excluding the blood glucose reference sample further comprises excluding said blood glucose reference sample in response to detecting at least one contaminant in said blood glucose reference sample. In one example, detecting the at least one contaminant further comprises detecting the contaminant based, at least in part, on at least one detected signature associated the said contaminant. In one example, the signature is based, at least in part, on a residue profile. In another example, the signature is based, at least in part, on a time to peak in a current signal generated in response to the blood glucose reference sample. In another example, the signature is based, at least in part, on a spectral analysis of a current signal generated in response to the blood glucose reference sample.

In another example implementation, the method further comprises: characterizing said trend based, at least in part, on a computed rate of change associated with glucose sensor measurements, and wherein determining whether said blood glucose reference sample deviates from said trend further comprises: comparing the blood glucose reference sample with a temporally associated glucose sensor measurement; and selectively excluding the blood glucose reference sample value based, at least in part, on the comparison. In another sample implementation, selectively excluding a blood glucose reference sample further comprises: if said computed rate of change exceeds a positive threshold, selectively excluding said blood glucose reference sample if a difference between the blood glucose reference sample and the temporally associated glucose sensor measurement exceed a threshold. In another implementation, selectively excluding the blood glucose reference sample further comprises: if said computed rate of change is less than a negative threshold, selectively excluding the blood glucose reference sample if a difference between the blood glucose reference sample and the temporally associated glucose sensor measurement exceed a threshold.

In another particular implementation, an apparatus comprises: a glucose sensor; and a processor to: receive a signal generated in response to a blood glucose reference sample obtained from a patient; and based, at least in part on predefined criteria, selectively exclude use of the received signal in deriving a relationship between signal values generated by said glucose sensor and blood glucose sensor measurements based, at least in part, on a temporal pairing of the blood glucose reference sample with at least one glucose sensor signal value. In one example implementation, the received signal comprises a current signal generated in response to a concentration of glucose in said blood glucose reference sample. In another implementation, the processor further to generate an alarm signal in response to said exclusion of the received signal.

In another particular implementation, an article comprises: a non-transitory storage medium comprising machine-readable instructions stored thereon which are executable by a special purpose computing apparatus to: receive a signal generated in response to a blood glucose reference sample obtained from a patient; and based, at least in part on predefined criteria, selectively exclude use of the received signal in deriving a relationship between signal values generated by a glucose sensor and blood glucose sensor measurements based, at least in part, on a temporal pairing of said blood glucose reference sample with glucose sensor signal values. In an example implementation, the instructions are further executable by the special purpose computing apparatus to: determine that said blood glucose reference sample deviates from a trend. In another example implementation, the instructions are further executable by the special purpose computing apparatus to determine that said blood glucose reference sample deviates from the trend by: correlating said blood glucose reference sample with a glucose sensor measurement; and determining whether a relative difference between a value of the blood glucose reference sample and the glucose sensor measurement exceeds a threshold. In another example implementation, the instructions are further executable by the special purpose computing apparatus to exclude said blood glucose reference sample in response to detection of at least one contaminant in said blood glucose reference sample. In yet another example implementation, the instructions are further executable by said special purpose computing apparatus to detect said at least one contaminant based, at least in part, on at least one detected signature associated with said contaminant.

In another particular implementation, an apparatus comprises: means for obtaining a blood glucose reference sample from a patient; based, at least in part on predefined criteria, means for selectively excluding the blood glucose reference sample for use in deriving a relationship between glucose sensor signal values and glucose sensor measurements based, at least in part, on temporal pairings of at blood glucose reference samples with glucose sensor signal values.

Other alternative example embodiments are described herein and/or illustrated in the accompanying Drawings. Additionally, particular example embodiments may be directed to an article comprising a storage medium including machine-readable instructions stored thereon which, if executed by a special purpose computing device and/or processor, may be directed to enable the special purpose computing device/processor to execute at least a portion of described method(s) according to one or more particular implementations. In other particular example embodiments, a sensor may be adapted to generate one or more signals responsive to a measured blood glucose concentration in a body while a special purpose computing device/processor may be adapted to perform at least a portion of described method(s) according to one or more particular implementations based upon one or more signals generated by the sensor.

### DETAILED DESCRIPTION

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments.

Systems for monitoring glucose in the body, for the treatment of diabetes for example, typically employ one or more glucose sensors to measure a blood-glucose concentration. For example, such sensors may be adapted to generate one or more electrical signals having a value (e.g., voltage and/or current level) that is responsive to a concentration of glucose in a fluid such as blood or interstitial fluid. Such a measurement of a blood-glucose concentration may then be used for any one of several applications such as, for example, monitoring a blood-glucose concentration for a diabetes patient.

Over time and/or with normal wear and usage of a glucose sensor, such a relationship between a value of a signal generated by the glucose monitoring blood sensor and actual measured blood glucose concentration may change. Accordingly, calibration of the signal generated by such a glucose monitoring with reference samples of blood-glucose concentration may enable an accurate estimate of a relationship between signal values generated by a glucose sensor and blood-glucose concentration in a patient, leading to more effective applications of glucose sensors and better treatment of diabetes patients.

As shown in the drawings for purposes of illustration, embodiments are directed to calibration methods for a glucose monitor that is coupled to a sensor set to provide continuous data recording of readings of glucose levels from a sensor for a period of time. In one particular implementation, a sensor and monitor provide a glucose sensor and a glucose monitor for observing glucose levels in the blood and/or bodily fluids of a user. However, it will be recognized that further embodiments may be used to observe the levels of other body characteristics including, for example, analytes or agents, compounds or compositions, such as hormones, cholesterol, medications concentrations, viral loads (e.g., HIV), bacterial levels, or the like without deviating from claimed subject matter. In particular implementations, a glucose sensor is primarily adapted for use in subcutaneous human tissue. However, in still further embodiments, one or more sensors may be placed in other tissue types, such as muscle, lymph, organ tissue, veins, arteries or the like, and used in animal tissue to measure body characteristics. Embodiments may record readings from the sensor on an intermittent, periodic, on-demand, continuous, or analog basis.

As discussed below, a blood glucose sensor is typically calibrated with metered blood glucose reference samples which may be presumed to provide accurate measurements of blood glucose concentration in a patient. However, patients obtaining metered blood glucose reference samples (e.g., using a finger stick) typically do not have clean hands, which may lead to a contamination of a blood glucose reference sample which affects the accuracy of a desired measurement of blood glucose concentration. Other ambient contaminants may also affect the accuracy of a measurement derived from a blood glucose reference sample.

Briefly, in one particular embodiment, a metered blood glucose sample may be selectively accepted or rejected for use in calibrating a blood glucose sensor based, for example, on a sensor trend. Here, in one particular implementation, a metered blood glucose reference sample value may be compared with a sensor trend to determine consistency. However, this is merely an example, embodiment and claimed subject matter is not limited in this respect.

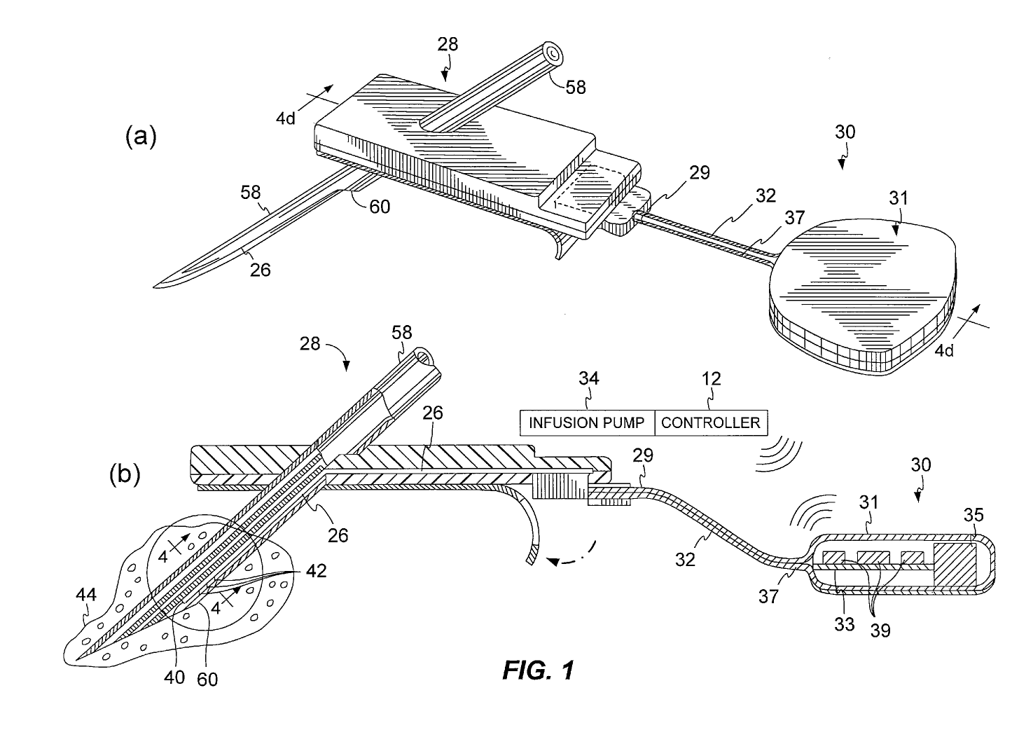

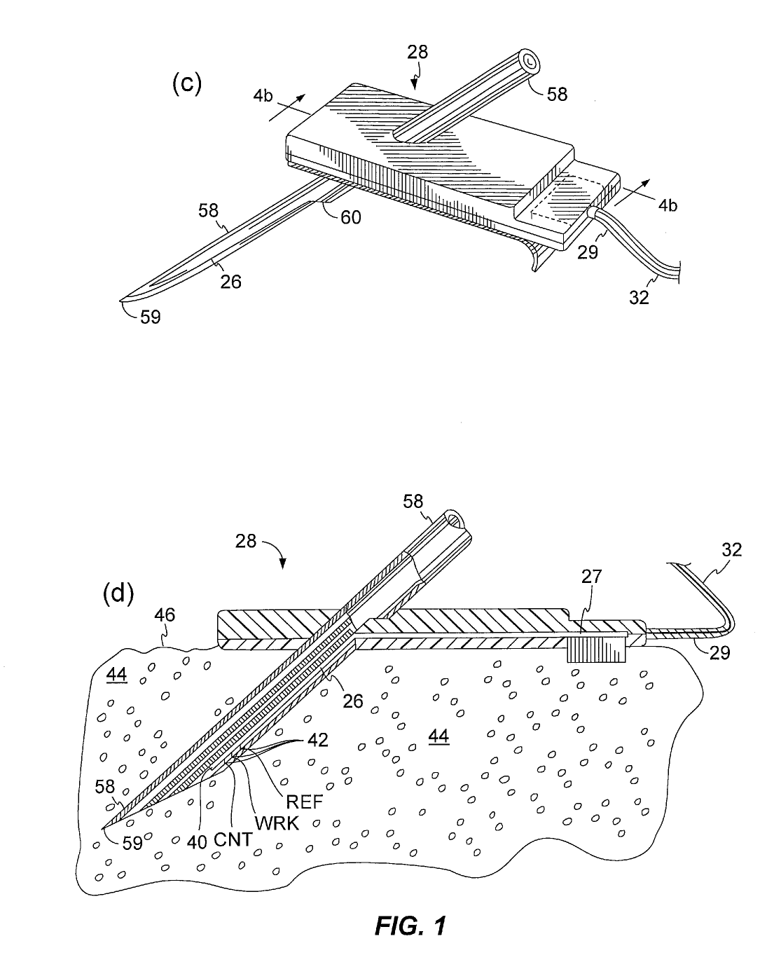

FIGS. 1(a)-1(d) illustrate a glucose monitor system for use with calibration methods described herein. Such a glucose monitor system, in accordance with one particular implementation, includes a subcutaneous glucose sensor set 28 and a glucose monitor 30. Here, glucose monitor 30 may be of the type described in U.S. Patent Application Ser. No. 60/121,664, filed on Feb. 25, 1999, entitled “Glucose Monitor System.” In alternative embodiments, the glucose monitor is of the type described in U.S. Pat. No. 7,324,012.

In one particular application, glucose monitor 30 may be worn by a user while connected to a surface mounted glucose sensor set 28 attached to the user's body by an electrically conductive cable 32, of the type described in U.S. Patent Application Ser. No. 60/121,656, filed on Feb. 25, 1999, entitled “Test Plug and Cable for a Glucose Monitor.” In one embodiment, a sensor interface may be configured in the form of a jack to accept different types of cables that provide adaptability of the glucose monitor 30 to work with different types of subcutaneous glucose sensors and/or glucose sensors placed in different locations of a user's body. However, in alternative embodiments, such a sensor interface may be permanently connected to the cable 32. In additional alternative embodiments, a characteristic monitor may be connected to one or more sensor sets to record data of one or more body characteristics from one or more locations on or in a user's body.

According to an embodiment, glucose sensor set 28 may be of a type described in U.S. Patent Application Ser. No. 60/121,655, filed on Feb. 25, 1999, entitled “Glucose Sensor Set”, or U.S. patent application Ser. No. 08/871,831, filed on Jun. 9, 1997, entitled “Insertion Set For A Transcutaneous Sensor.” Glucose sensor 26 may be of a type described in U.S. patent application Ser. No. 09/101,218, filed on Feb. 25, 1999, entitled “Glucose Sensor”, or described in commonly assigned U.S. Pat. Nos. 5,390,671; 5,391,250; 5,482,473; and 5,586,553; extends from the glucose sensor set 28 into a user's body with electrodes 42 of the glucose sensor 26 terminating in the user's subcutaneous tissue. See also U.S. Pat. No. 5,299,571. However, in alternative embodiments, glucose sensor 26 may use other types of sensors, such as chemical based, optical based, or the like. In further alternative embodiments, sensors may be of a type that is used on the external surface of the skin or placed below the skin layer of the user for detecting body characteristics.

According to an embodiment, glucose monitor 30 may be capable of recording and storing data as it is received from glucose sensor 2, and may include either a data port (not shown) or wireless transmitter and/or receiver (also not shown) for transferring data to and/or from a controller 12 such as a computer, communication station, a dedicated processor designed specifically to work with the glucose monitor, or the like. In a particular implementation, glucose monitor 30 may comprise a glucose monitor as described in U.S. Pat. No. 7,324,012.

In particular applications, a glucose monitor system may reduce inconvenience by separating complicated monitoring process electronics into two separate devices; the glucose monitor 30, which attaches to the glucose sensor set 28; and controller 12, which contains the software and programming instructions to download and evaluate data recorded by glucose monitor 30. In addition, the use of multiple components (e.g., glucose monitor 30 and controller 12) may facilitate upgrades or replacements, since one module, or the other, can be modified, re-programmed, or replaced without requiring complete replacement of a monitor system. Further, the use of multiple components can improve the economics of manufacturing, since some components may require replacement on a more frequent basis, sizing requirements may be different for each module, different assembly environment requirements, and modifications can be made without affecting the other components.

Glucose monitor 30 may take raw glucose sensor data from glucose sensor 26 and assess such sensor data in real-time and/or store it for later processing or downloading to controller 12, which in turn may analyze, display, and log the received data. Controller 12 may utilize the recorded data from glucose monitor 30 to analyze and review a blood glucose history. In particular embodiments, glucose monitor 30 is placed into a corn-station which facilitates downloading data to a personal computer for presentation to a physician. Software may be used to download such data, create a data file, calibrate the data, and display such data in various formats including charts, forms, reports, graphs, tables, lists and/or the like. In further embodiments, a glucose monitor system as described herein may be used in a hospital environment and/or the like.

In alternative embodiments, glucose monitor 30 may include at least portions of the software described as contained within controller 12 above. Glucose monitor 30 may further contain software enabling calibration of glucose sensor signals, display of a real-time blood glucose value, a showing of blood glucose trends, activate alarms and the like. A glucose monitor with these added capabilities is useful for patients that might benefit from real-time observations of their blood glucose characteristics even while they're not in close proximity to a computer, communication device and/or dedicated independent data processor.

Controller 12 may include a display (not shown) adapted to display calculated results of raw glucose sensor data received via a download from glucose monitor 30. Results and information displayed may include, but is not limited to, trending information of a characteristic (e.g., rate of change of glucose), graphs of historical data, average characteristic levels (e.g., glucose), stabilization and calibration information, raw data, tables (showing raw data correlated with the date, time, sample number, corresponding blood glucose level, alarm messages, and more) and/or the like. Such a display may also be used in conjunction with buttons (not shown) on controller 12, computer, communication station, characteristic monitor and/or or the like, to program or update data.

Glucose monitor 30 may be combined with other medical devices to accept other patient data through a common data network and/or telemetry system. Glucose monitor 30 may be combined with a blood glucose meter to directly import or correlate glucose calibration reference values such as described in U.S. patent application Ser. No. 09/334,996, filed Jun. 17, 1999, entitled “Characteristic Monitor With A Characteristic Meter and Method Of Using The Same.” Glucose monitor 30 may also be combined with semi-automated medication infusion pumps of the external type, as described according to particular embodiments in U.S. Pat. Nos. 4,562,751; 4,678,408; and 4,685,903; or automated implantable medication infusion pumps, as described according to particular embodiments in U.S. Pat. No. 4,573,994. Glucose monitor 30 may record data from the infusion pumps and/or may process data from both the glucose sensor 26 and an infusion pump to establish a closed loop system to control the infusion pump based, at least in part, on glucose sensor measurements. In other embodiments, other body characteristics are monitored, and the monitor may be used to provide feedback in a closed loop system to control a drug delivery rate. In further alternative embodiments, glucose monitor 30 can be combined with a glucose sensor set 28 as a single unit.

Glucose sensors may be replaced periodically to avoid infection, decaying enzyme coating and therefore sensor sensitivity, deoxidization of the electrodes, and/or the like. Here, a user may disconnect glucose sensor set 28 from cable 32 and glucose monitor 30. A needle 58 may be used to install another glucose sensor set 28, and then the needle 58 may be removed. Further description of the needle 58 and sensor set 28 according to particular embodiments may be found in U.S. Pat. Nos. 5,586,553; 6,368,141 and 5,951,521.

An initial reading may be downloaded from the glucose sensor set 10 and glucose monitor 30 to controller 12, to verify proper operation of glucose sensor 26 and glucose monitor 30. In particular embodiments, glucose sensor set 28 may provide data to glucose monitor 30 for one to seven days before replacement. Glucose sensor 26 may last in the user's body for longer or shorter periods of time depending on the quality of the installation, cleanliness, the durability of the enzyme coating, deoxidization of the sensor, user's comfort, and the like.

After installation into the body, glucose sensor 26 may be initialized to achieve a steady state of operation before starting a calibration process (e.g., for determining a function mapping sensor sample values with blood-glucose concentration measurements as discussed throughout).

The use of an initialization process can reduce the time for glucose sensor 26 stabilization from several hours to an hour or less, for example. One particular initialization procedure uses a two step process. First, a high voltage (e.g., between 1.0-1.1 volts—although other voltages may be used) may be applied between electrodes 42 of the sensor 26 for one to two minutes (although different time periods may be used) to allow sensor 26 to stabilize. Then, a lower voltage (e.g., between 0.5-0.6 volts—although other voltages may be used) may be applied for the remainder of the initialization process (e.g., 58 minutes or less). Other stabilization/initialization procedures using differing currents, currents and voltages, different numbers of steps, or the like, may be used. Other embodiments may omit such an initialization/stabilization process, if not required by a particular body characteristic sensor or if timing is not a factor. Alternatively, a characteristic monitor or controller 12 may apply an algorithm to the sensor data to determine whether initial transients have sufficiently diminished and the sensor is at a significantly stable state to begin calibration.

In particular embodiments, data may not be considered valid until a sensor initialization event flag (ESI) is set in data indicating that stabilization is complete. In one particular implementation, stabilization may be complete after 60 minutes or when a user enters a sensor initialization flag using one or more buttons on the glucose monitor 30. Following completion of stabilization/initialization, glucose monitor 30 may be calibrated to accurately interpret readings from the newly installed glucose sensor 26.

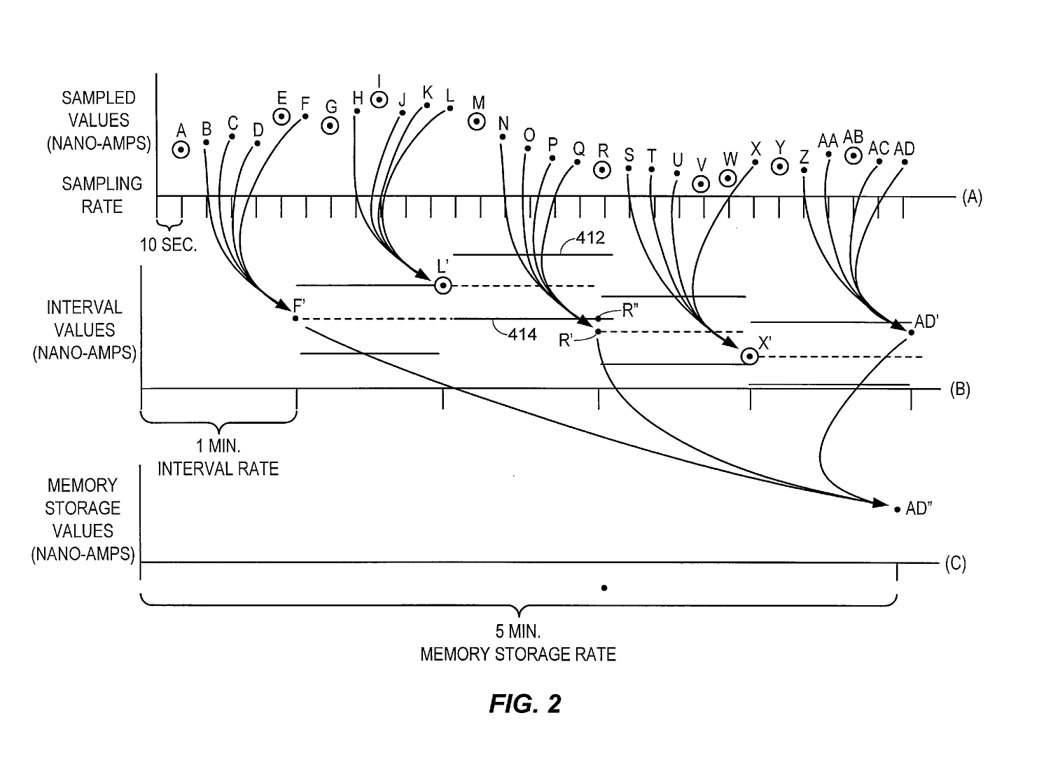

Beginning with the stabilization process, glucose monitor 30 may measure a continuous electrical current signal (ISIG) generated by glucose sensor 26 in response to a concentration of glucose present in the subcutaneous tissue of the user's body. In particular embodiments, glucose monitor 30 may sample the ISIG from glucose sensor 26 at a sampling rate of once every 10.0 seconds, for example, as shown in FIGS. 2a-c. Examples of sampled values are labeled A-AD in FIG. 2a. At an interval rate of once per minute, the highest and lowest of the sampled values (shown in FIG. 2a as circled sampled values A, E, G, I, M, R, V, W, Y, and AB) are ignored, and the remaining four sampled values from an interval are averaged to create interval values (shown in FIG. 2b as values F′, R′, X′, and AD′). At a glucose monitor memory storage rate of once every 5.0 minutes, the highest and lowest of the interval values (shown in FIG. 2b as values L′ and X′) are ignored and the remaining three interval values are averaged and stored in a glucose monitor memory as memory values (shown in FIG. 2c as point AD″). The memory values are retained in memory and may be downloaded to controller 12. Such memory values may be used to calibrate glucose monitor 30 and/or controller 12 and to analyze blood glucose levels. The sampling rate, interval rate and the memory storage rate may be varied as necessary to capture data with sufficient resolution to observe transients or other changes in the data depending on the rate at which sensor values can change, which is affected by the sensor sensitivity, the body characteristic being measured, the physical status of the user, and the like. In other embodiments, all of the sampled values may be included in the average calculations of memory storage values. In alternative embodiments, more or less sampled values or interval values are ignored depending on the signal noise, sensor stability, or other causes of undesired transient readings. Finally, in still other embodiments, all sampled values and/or interval values are stored in memory.

Clipping limits may be used to limit a signal magnitude variation from one value to the next thereby reducing the effects of extraneous data, outlying data points, or transients. In particular embodiments, clipping limits may be applied to interval values. For instance, interval values that are above a maximum clipping limit or below a minimum clipping limit may be replaced with the nearest clipping limit value.

In alternative embodiments, interval values that are outside of clipping limits may be ignored and not used to calculate a subsequent memory storage value. In particular implementations, detection of interval values outside of clipping limits may be considered a calibration cancellation event. In further particular embodiments, a calibration cancellation event may be recognized if more than one value is deemed outside of clipping limits.

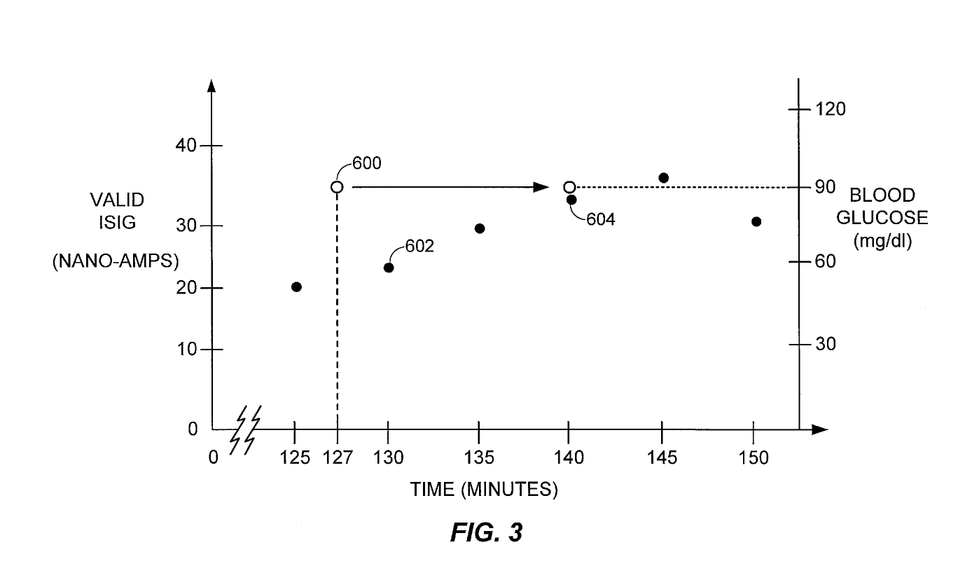

In a particular embodiment, a memory storage value may be considered valid (Valid ISIG value) unless one of the following calibration cancellation events occurs: an unstable signal alarm (as discussed above); a sensor initialization event (as discussed above); a sensor disconnect alarm; a power on/off event; an out-of-range alarm (as discussed above); or a calibration error alarm. Here, only Valid ISIG values may be used to calculate blood glucose levels by the glucose monitor 30 or controller 32, as shown in FIG. 3. Once a calibration cancellation event occurs, successive memory storage values are not valid, and therefore are not used to calculate blood glucose, until glucose monitor 30 or controller 32 is re-calibrated. If glucose monitor 30 is turned off for a short enough period of time, up to 30 minutes for example, memory storage values may be considered Valid ISIG values as soon as the power is restored. If the power is off for longer than 30 minutes, for example, glucose monitor 30 may be re-calibrated before ISIG values are considered valid. Alternatively, power may be off for a duration such as 30 minutes or longer and, once power is restored, the memory storage values may comprise Valid ISIG values. Here, a sensor disconnect alarm may be activated if the glucose monitor 30 does not detect a signal. In particular embodiments, if two or more out of five interval values collected within a given memory storage rate are less than 1.0 nA, a disconnect alarm may be triggered. In alternative embodiments, greater or fewer values need be below a particular threshold current level to trigger a disconnect alarm depending of the acceptable range or sensor readings and the stability of an associated sensor signal. Two remaining calibration cancellation events, the calibration error and an alternative embodiment for the out-of-range alarm, are discussed in conjunction with the calibration process below.

Particular implementations are directed to calibration techniques that may be used by either glucose monitors during real-time measurements of one or more signals from a glucose sensor, or post processors during post-processing of data that has been previously recorded and downloaded.

To calibrate glucose monitor 30, a function mapping sensor signal values (e.g., Valid ISIG values) to blood-glucose sensor measurements may be determined. As discussed above, such a function may comprise a non-linear function. For example, calibration factor called a sensitivity ratio (SR) (blood glucose level/Valid ISIG value) may be calculated for a particular glucose sensor 26. The SR may represent a calibration factor used to measure/estimate a blood glucose concentration in certain cases based, at least in part on a Valid ISIG value (Nano-Amps) into a blood glucose level (mg/dl or mmol/l). In alternative embodiments, units for the SR may vary depending on a type of signal available from the sensor (frequency, amplitude, phase shift, delta, current, voltage, impedance, capacitance, flux, and the like), the magnitude of the signals, the units to express the characteristic being monitored, and/or the like.

In particular implementations, a user may obtain a blood glucose reference reading from a common glucose meter, or another blood glucose measuring device, and immediately enter such a blood glucose reference reading into glucose monitor 30. Such a blood glucose reference reading is assumed to be accurate and is used as a reference for calibration. Glucose monitor 30, or a controller 12, may temporally correlate a blood glucose reference reading with a Valid ISIG value to establish a “paired calibration data point.” Since a glucose level in an interstitial body fluid tends to lag behind a blood glucose level, glucose monitor 30 or controller 12 applies a delay time and then pairs the blood glucose reference reading with a Valid ISIG value as shown in FIG. 3. In particular embodiments, an empirically derived ten minute delay may be used. In a particular implementation where Valid ISIG values are averaged and stored every five minutes, glucose monitor 30 may correlate a blood glucose reference reading with the third Valid ISIG stored in memory after the blood glucose reference reading is entered (resulting in an effective delay of ten to fifteen minutes in this particular example). FIG. 3 illustrates an example, in which a blood glucose reference reading 600 of 90 mg/dl is entered into glucose monitor 30 at 127 minutes. The next Valid ISIG value 602 may be stored at 130 minutes. Given a 10 minute delay, a glucose reference reading 600 may be paired with Valid ISIG value 604 which is stored at 140 minutes with a value of 30 Nano-amps. Note that two numbers are needed to establish one paired calibration data point, a blood glucose reference reading and a Valid ISIG.

Other delay times may be used depending on a particular user's metabolism, response time of the sensor, delay time incurred for the glucose meter to calculate a reading and for the reading to be entered into the glucose monitor 100, a type of analyte being measured, the tissue that the sensor is placed into, environmental factors, whether the previous glucose Valid ISIG value (or the trend of the Valid ISIG values) was higher or lower than current Valid ISIG value, and/or the like. Once paired calibration data is available, an appropriate calibration process may be applied dependent on how many paired calibration data points are available since the last calibration, the total period of time that glucose sensor 26 has been in use, and the number of times glucose sensor 26 has been calibrated.

In particular embodiments, blood glucose reference readings may be entered into glucose monitor 30 periodically throughout each day of use to, among other things, calibrate glucose sensor 26. Techniques to calibrate a glucose sensor may be found in U.S. patent application Ser. Nos. 12/345,477, filed Dec. 29, 2008, and 13/239,265, filed on Sep. 21, 2011, which are assigned to the assignee of subject matter claimed herein and incorporated herein by reference. Here, calibration may be conducted immediately after the initialization/stabilization of glucose sensor 26 and once a day thereafter. However, such calibration may be conducted more or less often depending on whether glucose sensor 26 has been replaced, whether a calibration cancellation event has occurred, the stability of glucose sensor 26 sensitivity over time, and/or the like.

In particular embodiments, blood glucose reference readings may be collected several times per day while a new calibration factor is calculated only once per day. Therefore, more than one paired calibration data point may be collected between calibrations. In alternative embodiments, the glucose monitor is calibrated every time a new paired calibration data point is collected.

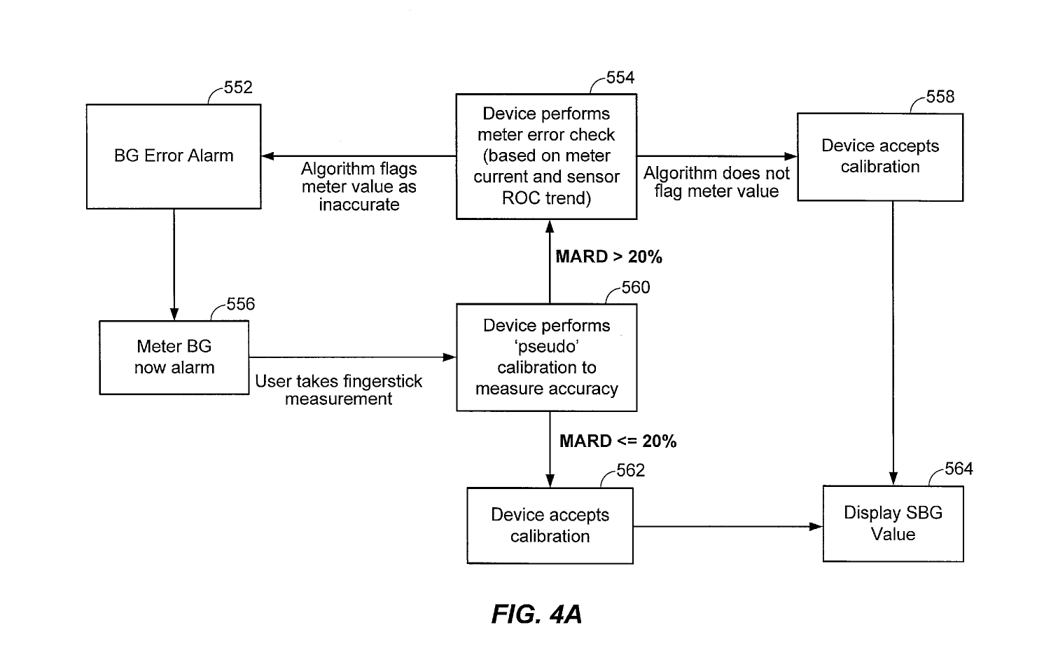

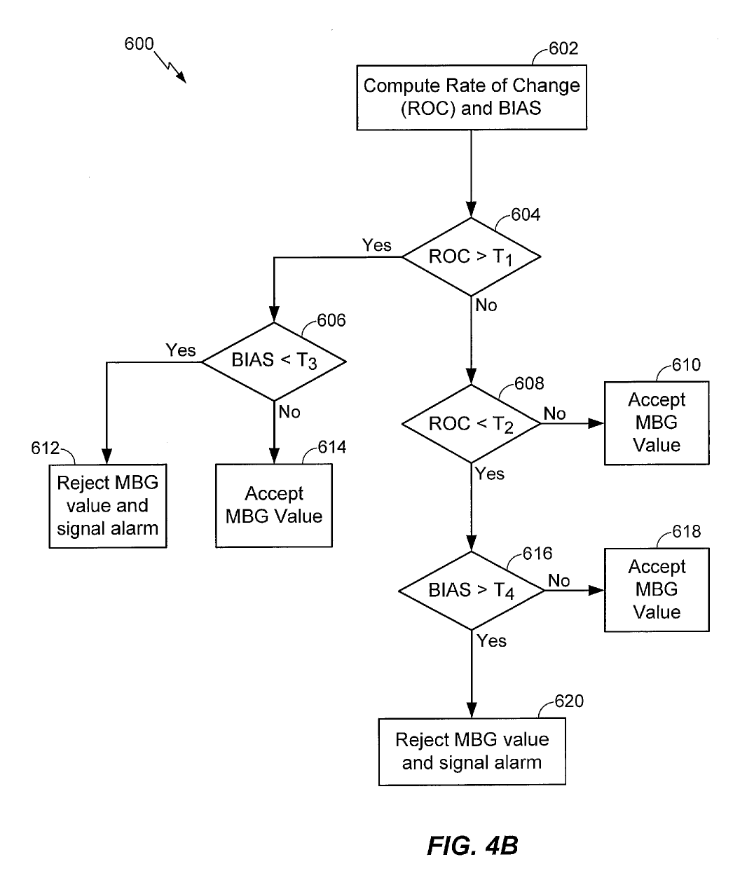

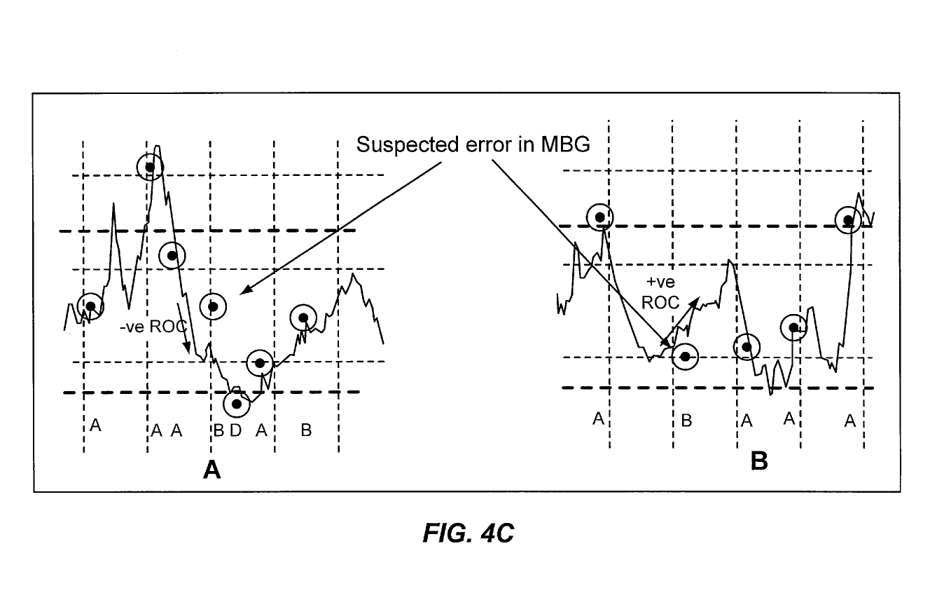

As discussed above, values for ISIG may be validated prior to use in a calibration process. In particular implementations, metered blood glucose reference samples may be selectively accepted or rejected for use in a process to calibrate a blood glucose sensor (e.g., estimate one or more parameters defining a linear relationship between ISIG and a concentration of glucose in blood). FIG. 4A is a flow diagram of a process for incorporating metered blood glucose reference samples to calibrate a blood glucose sensor according to an embodiment. At block 560, a pseudo calibration process is performed by pairing a new metered blood glucose reference sample obtained at block 556 with a valid ISIG value as discussed herein to provide a slope and offset to characterize a function mapping ISIG values to sensor blood glucose measurements. A mean absolute relative difference (MARD) may then be computed as follows:

MARD=100×(MBG−SBG)/MGB,

Where:
MBG is a blood glucose concentration value obtained from a metered blood glucose reference sample; and
SBG is a sensor blood glucose concentration measurement based upon application of an ISIG value to a resulting calibration operation.

In a particular implementation, if MARD is less than a threshold percentage (e.g., MARD 20% as shown in the particular non-limiting example of FIG. 4A), then the calibration operation performed at block 560 is accepted at block 562 and a computed SBG value may be displayed at block 564. If MARD is less than the threshold percentage, however, block 554 may conduct additional analysis to evaluate the metered blood glucose reference sample before acceptance. In one embodiment, the MBG sample may be determined to be within a standard deviation of a mean value. While the particular example implementation of FIG. 4A applies a 20% threshold to MARD to determine whether a blood glucose reference sample may be accepted or further analyzed, it should be understood that larger or smaller thresholds may be applied without deviating from claimed subject matter. FIG. 4B is flow diagram of a process to selectively include or exclude a metered blood glucose reference sample according to a particular implementation of block 554 of FIG. 4A. Here, a metered blood glucose reference sample may be selectively excluded if one or more conditions are satisfied. In the particular implementation of FIG. 4B, a metered blood glucose reference sample may be excluded if it is inconsistent with a rate of change associated with computed SBG values.

As pointed out above, one condition set forth in FIG. 4A is based upon a comparison of an MBG sample value against a rate of change trend for sensor measurements. In one implementation, an expected value for ISIG temporally correlated or paired with a current MBG sample may be obtained, for example, from a look up table. A rate of change vector may then be determined as illustrated in the particular examples of FIG. 4C. Here, if a rate of change vector is positive and has a magnitude that exceeds a threshold, and if the MBG sample value obtained at block 556 is smaller than the SBG value obtained during pseudo calibration at block 560, the MBG sample value may be rejected. Likewise, if a rate of change vector is negative and has a magnitude that exceeds a threshold, and if the MBG sample value obtained at block 556 is greater than the SBG value obtained during pseudo calibration at block 560, the MBG sample value may be rejected.

FIG. 4B is a flow diagram of a process 600 for selectively validating an MBG sample value even in conditions where an MARD between the MBG value and an SBG value exceeds a threshold (e.g., at block 554). In one embodiment, an SBG measurement may be taken at regular intervals such as once every five minutes. Other alternative regular intervals may be used without deviating from claimed subject matter. Block 602 may from time to time compute a bias and a rate of change in SBG values from time to time using any one of several techniques. A bias value may be computed as follows:

BIAS=MBG−SBG.

In a particular implementation, a rate of change may be computed as an instantaneous rate of change as follows:

ROCᵢ=Σₙ₌₁¹[SBG₍ᵢ₋ₙ₊₁₎−SBG₍ᵢ₋ₙ₎],

Where:
ROCᵢ is a rate of change; and
SBGₖ is the sensor blood glucose measurement obtained for interval k.

In an alternative implementation, and to allow for the cancellation of noise, a rate of change may be computed over three SBG measurement intervals (e.g., 15 minutes for five minute intervals) as follows:

ROCᵢ=Σₙ₋₁³[SBG₍ᵢ₋ₙ₊₁₎−SBG₍ᵢ₋ₙ₎].

In yet another alternative implementation, a rate of change may be computed over fifteen SBG measurement intervals (e.g., 15 minutes for one minute intervals) as follows:

ROCᵢ=Σₙ₌₁¹⁵[SBG₍ᵢ₋ₙ₊₁₎−SBG₍ᵢ₋ₙ₎].

Based at least in part on values for BIAS and ROC computed at block 602, process 600 may selectively accept or reject an MBG value for use in calibration of a glucose sensor. Here, thresholds may be applied to computed values of BIAS and ROC to determine whether an MBG value is to be accepted. In the particularly illustrated embodiment, threshold T₁ may comprise a positive value while T₂ may comprise a negative value. If a computed ROC is between, is between T₁ and T₂, as determined at diamonds 604 and 608, the MBG value may be accepted at block 610. If ROC exceeds T₁ as determined at diamond 604 and BIAS is less than a threshold T₃ (representing a negative value), the MBG value may be rejected at block 612 and an alarm may be triggered. Similarly, if ROC is less than T₂ and BIAS exceeds a threshold T₄ (representing a positive value), the MBG value may be rejected at block 620 and an alarm may be triggered. Otherwise, the MBG value may be accepted at block 614 or 618. In particular implementations, values for T₁, T₂, T₃ and T₄ may be determined based, at least in part, on a desired tradeoff between sensitivity/accuracy of calibration of a glucose sensor and tolerance for false alarms leading to obtaining unnecessary additional blood glucose reference samples. For example, values for T₁, T₂, T₃ and T₄ may be determined using experimentation, and trial and error.

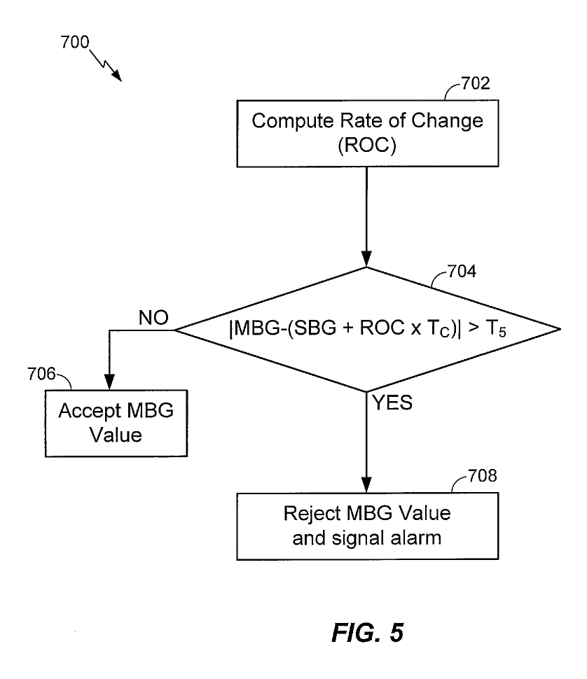

FIG. 5 illustrates a process for determining whether to accept or reject a blood glucose reference sample for use in calibration of a blood glucose sensor according to an alternative implementation of block 554 of FIG. 4A. The particular implementation of FIG. 5 may address a lag between changes in a blood glucose concentration and subsequent changes of a glucose concentration observable in interstitial fluid. Here, diamond 704 compares an MBG values with a predicted sensor blood glucose value expressed as SBG+ROC×T_{c}, where T_{c} a rate of change (ROC) may be computed at block 702 as discussed above. If a difference between MBG and the predicted sensor blood glucose value exceeds a threshold T₅, block 708 may reject the MBG value and signal an alarm. Otherwise, if the difference MBG and the predicted sensor blood glucose value does not exceed threshold T₅, block 706 may accept the MBG value.

In other embodiments, in addition to or instead of a rate of change analysis as illustrated in FIGS. 4B and 5, block 554 may analyze the MBG sample obtained at 556 using analyte detection to detect the presence of contaminants. For example, process 600 or 700 may be apply an additional test to confirm an absence of contamination in an MBG sample before accepting the MGB value at any of blocks 610, 614 or 618 of process 600, or block 706 of process 700. Here, analysis may be applied to detect a signature indicating a presence of predetermined analyte contaminants based upon feature recognition. In one implementation, acceptance of an MBG value at blocks 610, 614, and 618 of process 600 or block 706 of process 700 may further require a determination of an absence of contaminants in the MBG sample. Here, a presence of a particular contaminant may be detected using any one or a combination of techniques including, for example, a residue profile analysis, initial response analysis or a power spectral density analysis.

In one particular implementation, a blood glucose meter may provide to controller 12 with a decaying current signal which is to characterize a blood glucose reference sample. This current sample may be processed to detect features indicating a presence of a contaminant using one or more of the above identified techniques. In one implementation, a patient's meter signal profile may be developed based, at least in part, on an initial meter point including, for example, setting limits for tolerances. Such an initial meter point may be presumed to be substantially free of contaminants.

In a particular implementation, a residue profile for an MBG sample may be detected based upon a previous blood glucose sample which is known to be clean. Here, a residue profile for an MBG sample may be isolated using any one of several techniques known in the art of signal processing. For example, subtracting a clean blood profile from a current profile of a new blood glucose reference sample may provide a residue profile, which may be indicative of a contaminant distorting the profile of an uncontaminated metered blood glucose reference sample.

An initial response determination may evaluate a slope of an initial response of a current signal generated from an MBG sample. Here, a slope of an initial response of a current signal generated from an MBG sample may be isolated using any one of several techniques known in the art of signal processing. For example, a time to peak of the current signal may be compared with a time to peak for a clean signal. Other techniques for analyzing an initial response may be used without deviating from claim subject matter. In addition to the residue profile analysis and initial response analysis, a spectral analysis may be applied to a current signal generated from the MBG sample. Also, a spectral analysis may be applied to a current signal received from a blood glucose meter. Here, a fast Fourier transform may be applied to the current signal and resulting power spectral densities may be analyzed. In particular, such a power spectral density may be analyzed to identify certain features such as particular peaks and nulls indicating a presence of a known contaminant. Other example techniques for performing a spectral analysis of a current signal generated from an MBG sample may be found at U.S. Pat. No. 7,090,764.

In a particular implementation, a presence of a contaminant may be detected using one or a combination of numerical indicators generated using the aforementioned initial residue profile, response, and spectral analysis techniques. These numerical indicators may be normalized, combined and compared with a threshold to determine whether or not a contaminant is present.

In addition, although particular process illustrated in figures include specific operations occurring in a particular order, in alternative embodiments, certain of these operations may be performed in a different order, modified, or removed while not deviating from claimed subject matter. Moreover, other operations may be added to and/or combined with the above described process without deviating from claimed subject matter.

Unless specifically stated otherwise, as apparent from the following discussion, it is appreciated that throughout this specification discussions utilizing terms such as “processing”, “computing”, “calculating”, “determining”, “estimating”, “selecting”, “weighting”, “identifying”, “obtaining”, “representing”, “receiving”, “transmitting”, “storing”, “analyzing”, “creating”, “contracting”, “associating”, “updating”, or the like refer to the actions or processes that may be performed by a computing platform, such as a computer or a similar electronic computing device, that manipulates or transforms data represented as physical, electronic or magnetic quantities or other physical quantities within the computing platform's processors, memories, registers, or other information storage, transmission, reception or display devices. Accordingly, a computing platform refers to a system or a device that includes the ability to process or store data in the form of signals. Thus, a computing platform, in this context, may comprise hardware, software, firmware or any combinations thereof. Further, unless specifically stated otherwise, a process as described herein, with reference to flow diagrams or otherwise, may also be executed or controlled, in whole or in part, by a computing platform.

It should be noted that, although aspects of the above system, method, or process have been described in a particular order, the specific order is merely an example of a process and claimed subject matter is of course not limited to the order described. It should also be noted that the systems, methods, and processes described herein, may be capable of being performed by one or more computing platforms. In addition, the methods or processes described herein may be capable of being stored on a storage medium as one or more machine readable instructions, that if executed may enable a computing platform to perform one or more actions. “Storage medium” as referred to herein relates to media capable of storing information or instructions which may be operated on, or executed by, by one or more machines. For example, a storage medium may comprise one or more storage devices for storing machine-readable instructions or information. Such storage devices may comprise any one of several media types including, for example, magnetic, optical or semiconductor storage media. For further example, one or more computing platforms may be adapted to perform one or more of the processed or methods in accordance with claimed subject matter, such as the methods or processes described herein. However, these are merely examples relating to a storage medium and a computing platform and claimed subject matter is not limited in these respects.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter may also include all aspects falling within the scope of appended claims, and equivalents thereof.

What is claimed is:

1. A method comprising:
obtaining a blood glucose reference sample from a patient;
based, at least in part on predefined criteria, selectively excluding the blood glucose reference sample for use in deriving a relationship between glucose sensor signal values and glucose sensor measurements based, at least in part, on temporal pairings of blood glucose reference samples with glucose sensor signal values.

2. The method of claim 1, and wherein said excluding said blood glucose reference sample further comprises:
determining that said blood glucose reference sample deviates from a trend.

3. The method of claim 2, wherein determining that said blood glucose reference sample deviates from the trend further comprises:
correlating said blood glucose reference sample with a glucose sensor measurement; and
determining whether a relative difference between a value of the blood glucose reference sample and the glucose sensor measurement exceeds a threshold.

4. The method of claim 1, wherein said excluding further comprises excluding said blood glucose reference sample in response to detecting at least one contaminant in said blood glucose reference sample.

5. The method of claim 4, wherein said detecting said at least one contaminant further comprises detecting based, at least in part, on at least one detected signature associated with said contaminant.

6. The method of claim 5, wherein the signature is based, at least in part, on a residue profile.

7. The method of claim 5, wherein said signature is based, at least in part, on a time to peak in a current signal generated in response to said blood glucose reference sample.

8. The method of claim 5, wherein said signature is based, at least in part, on a spectral analysis of a current signal generated in response to said blood glucose reference sample.

9. The method of claim 2, and further comprising:
characterizing said trend based, at least in part, on a computed rate of change associated with glucose sensor measurements, and wherein said determining whether said blood glucose reference sample deviates from said trend further comprises:
comparing said blood glucose reference sample with a temporally associated glucose sensor measurement; and
selectively excluding the blood glucose reference sample value based, at least in part, on said comparison.

10. The method of claim 9, wherein said selectively excluding further comprises:
if said computed rate of change exceeds a positive threshold, selectively excluding said blood glucose reference sample if a difference between the blood glucose reference sample and the temporally associated glucose sensor measurement exceed a threshold.

11. The method of claim 9, wherein said selectively excluding further comprises:
if said computed rate of change is less than a negative threshold, selectively excluding said blood glucose reference sample if a difference between the blood glucose reference sample and the temporally associated glucose sensor measurement exceed a threshold.

12. An apparatus comprising:
a glucose sensor; and
a processor to:
receive a signal generated in response to a blood glucose reference sample obtained from a patient; and
based, at least in part on predefined criteria, selectively exclude use of the received signal in deriving a relationship between signal values generated by said glucose sensor and blood glucose sensor measurements based, at least in part, on a temporal pairing of said blood glucose reference sample with at least one glucose sensor signal value.

13. The apparatus of claim 12, wherein said received signal comprises a current signal generated in response to a concentration of glucose in said blood glucose reference sample.

14. The apparatus of claim 12, the processor further to generate an alarm signal in response to said exclusion of the received signal.

15. An article comprising:
a non-transitory storage medium comprising machine-readable instructions stored thereon which are executable by a special purpose computing apparatus to:
receive a signal generated in response to a blood glucose reference sample obtained from a patient; and
based, at least in part on predefined criteria, selectively exclude use of the received signal in deriving a relationship between signal values generated by a glucose sensor and blood glucose sensor measurements based, at least in part, on a temporal pairing of said blood glucose reference sample with glucose sensor signal values.

16. The article of claim 15, wherein said instructions are further executable by said special purpose computing apparatus to:
determine that said blood glucose reference sample deviates from a trend.

17. The article of claim 16, wherein said instructions are further executable by said special purpose computing apparatus to determine that said blood glucose reference sample deviates from the trend by:
correlating said blood glucose reference sample with a glucose sensor measurement; and
determining whether a relative difference between a value of the blood glucose reference sample and the glucose sensor measurement exceeds a threshold.

18. The article of claim 15, wherein said instructions are further executable by said special purpose computing apparatus to exclude said blood glucose reference sample in response to detection of at least one contaminant in said blood glucose reference sample.

19. The article of claim 18, wherein said instructions are further executable by said special purpose computing apparatus to detect said at least one contaminant based, at least in part, on at least one detected signature associated with said contaminant.20.
An apparatus comprising:
means for obtaining a blood glucose reference sample from a patient;
based, at least in part on predefined criteria, means for selectively excluding the blood glucose reference sample for use in deriving a relationship between glucose sensor signal values and glucose sensor measurements based, at least in part, on temporal pairings of at blood glucose reference samples with glucose sensor signal values.
